(19) United States
(12) Patent Application Publication
(10) Pub. No.: US 2023/0113440 A1
(43) Pub. Date: Apr. 13, 2023

(54) LIQUID CRYSTAL SULFATE-FREE SHAMPOO

(71) Applicant: The Procter & Gamble Company, Cincinnati, OH (US)

(72) Inventors: Brooke Michele Cochran, Cincinnati, OH (US); Mark Anthony Brown, Union, KY (US); Debora W. Chang, Mason, OH (US)

(21) Appl. No.: 17/960,867

(22) Filed: Oct. 6, 2022

Related U.S. Application Data
(60) Provisional application No. 63/253,362, filed on Oct. 7, 2021.

(51) Int. Cl. A61K 8/81; A61K 8/02; A61K 8/46; A61K 8/44; A61K 8/49; A61Q 5/02; A61Q 5/12

(52) U.S. Cl. CPC A61K 8/817; A61K 8/0295; A61K 8/466; A61K 8/442; A61K 8/4926; A61Q 5/02; A61Q 5/12; A61K 2800/30; A61K 2800/262; A61K 2800/34

(57) ABSTRACT

A shampoo composition comprising a. 3% to 35% of an anionic surfactant, wherein the anionic surfactant is substantially free of sulfated surfactants; b. 3% to 15% of an amphoteric surfactant; and c. 0.01% to 2% of a cationic polymer having a charge density 2.0 to 10.0 meq/g; and wherein the composition is isotropic and forms a lyotropic liquid crystal coacervate upon dilution.

### FIELD

The present invention relates to a shampoo composition, in particular a conditioning shampoo composition that is substantially free of surfactants containing sulfates and which forms lyotropic liquid crystals upon dilution.

### BACKGROUND OF THE INVENTION

Consumers use shampoo to remove dirt and oil from the surface of hair fibers and scalp. In traditional shampoo compositions, this cleaning is generally provided by incorporating a surfactant system that contains sulfate-based anionic surfactants (e.g., sodium lauryl sulfate, sodium laureth sulfate) into the shampoo composition. These traditional shampoo compositions are easy to apply because they have a viscosity such that the shampoo can be dispensed into an open palm and then spread across the user's hair and scalp. Another advantage of shampoos with sulfate-based surfactants is that they can be paired with cationic polymers that form a coacervate when diluted with water during use that is deposited onto the hair to provide a conditioning benefit.

Recently, many consumers, especially those with color-treated hair, may prefer a shampoo with a sulfate-free surfactant system. These consumers may also want conditioning polymers in their shampoo because higher conditioning shampoos feel less stripping to the hair. However, formulating a shampoo that contains a sulfate-free anionic surfactant and a cationic conditioning polymer can be difficult due to the formation of an in situ coacervate which typically leads to formulation instability. In shampoo compositions with sulfate-free surfactants and cationic polymers, it is desirable for the composition to be isotropic, and thus not have an in situ coacervate form in the composition prior to use (rather than during use, which is desired). The in situ coacervate can separate resulting in inconsistent in-use performance and the product can appear cloudy and/or with a precipitated layer.

Lyotropic liquid crystal coacervates that form with high charge density cationic polymers can deliver superior hair conditioning and color retention benefits. In shampoos containing sulfate-based surfactants, the incorporation of high charge density cationic polymers inevitably results in the formation of lyotropic liquid crystals in the neat or undiluted product. But the presence of lyotropic liquid crystals in neat or undiluted product requires the addition of a suspension aid to prevent phase separation of the liquid crystals, a form of in situ coacervate, where the suspension aid adds cost and complexity to the formula and can reduce overall performance of the shampoo. Additionally, the presence of in situ coacervate prevents product clarity, which certain consumers prefer. Therefore, there is a need for a stable isotropic shampoo product with a superior product performance that contains a surfactant system that is substantially free of sulfate-based surfactants, that along with cationic polymers form lyotropic liquid crystal coacervate upon dilution without forming the in situ coacervate phase in the product prior to dilution with water.

### SUMMARY OF THE INVENTION

A shampoo composition comprising a. 3% to 35% of an anionic surfactant, wherein the anionic surfactant is substantially free of sulfated surfactants; b. 3% to 15% of an amphoteric surfactant; and c. 0.01% to 2% of a cationic polymer having a charge density 2.0 to 10.0 meq/g; and wherein the composition is isotropic and forms a lyotropic liquid crystal coacervate upon dilution.

### BRIEF DESCRIPTION OF THE DRAWINGS

FIG. 1 is a 20×micrograph of a shampoo composition that contains in situ lyotropic liquid crystal coacervate.

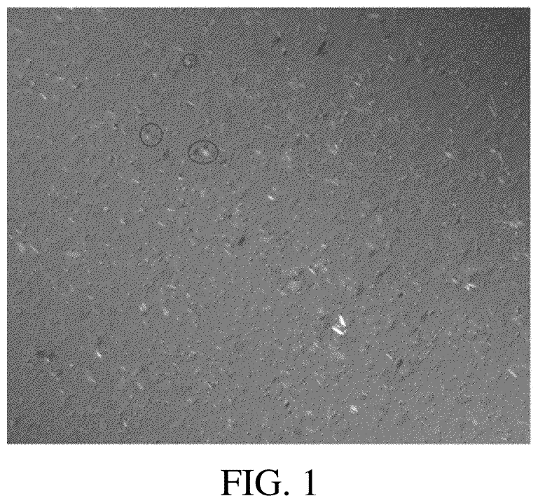

FIG. 2 is a 20×micrograph of the shampoo composition of FIG. 1 under cross-polarized light.

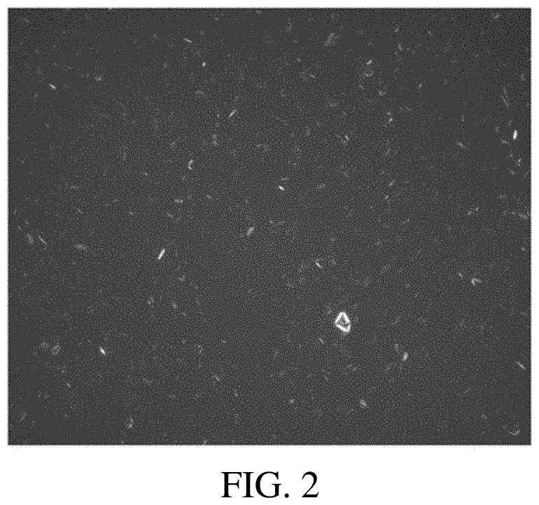

FIG. 3 is a picture of an isotropic sulfate-free shampoo (pre-dilution), which shows the absence of in situ coacervate.

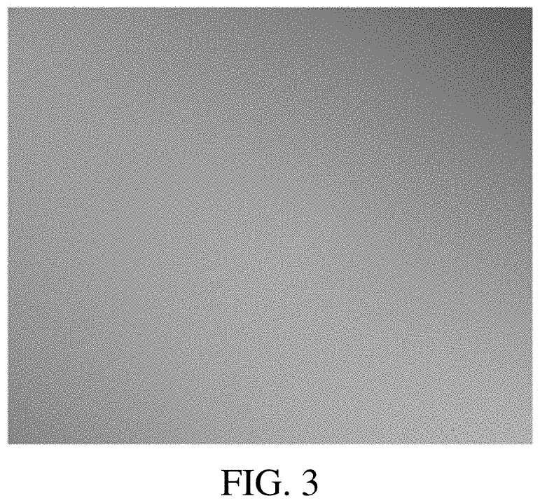

FIG. 4 is a micrograph of a diluted shampoo (10:1 water:shampoo) under cross-polarized light that shows a liquid crystal coacervate.

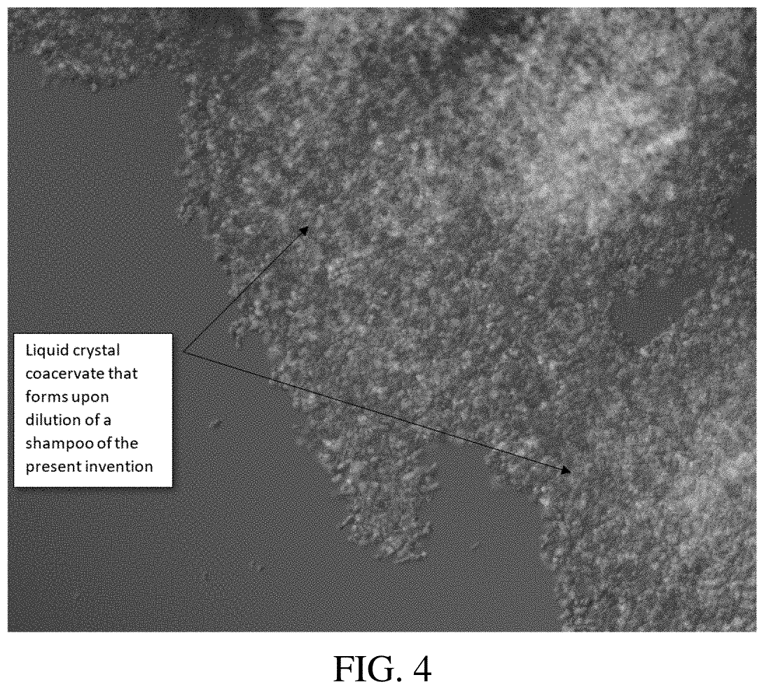

FIG. 5 shows the scattering patterns for Examples 2 and 4.

### DETAILED DESCRIPTION OF THE INVENTION

Sulfate-free shampoos tend to underperform versus sulfate-based shampoos for conditioning hair. Furthermore, damaged hair tends to be more difficult to condition than non-damaged hair, requiring additional hair conditioning to achieve the look and feel of hair that consumers desire. Increased levels of conditioning actives such as silicone and/or polymers can be used to help compensate for the hair conditioning challenges of a sulfate-free shampoo, which necessarily adds cost to the formula and the complexity of maintaining a stable product.

At least some consumers prefer a shampoo that uses a sulfate-free surfactant systems due to its perceived gentleness on hair. These consumers may also prefer shampoos with cationic conditioning polymers because they feel less stripping to the hair. However, such shampoos may exhibit instability due to the formation of in situ coacervate phase in the composition prior to use. Rather, the coacervate should be formed during use when the shampoo composition is diluted with water. The in situ coacervate that forms prior to dilution can cause inconsistent product performance, a cloudy appearance in the composition, and/or the formation of a precipitate layer.

The personal care compositions herein include a cationic polymer having a high charge density. Surprisingly it has been found that the cationic polymer, in combination with the anionic sulfate-free surfactant component, does not form lyotropic liquid crystals in neat or undiluted product but does form lyotropic liquid crystals upon dilution of the product. The cationic polymer can therefore be formulated in a stable isotropic personal care composition without the need for a suspending agent, while still providing the unique conditioning benefits of liquid crystals during use which includes improved wet hair conditioning with a clean rinse.

An added benefit of the compositions of the present invention is the reduction of the surface energy of damaged hair, particularly chemically treated hair, thereby increasing its hydrophobicity and restoring its natural smoothness and lubricious feel. The reduction of the hair's surface energy also improves silicone deposition efficiency, enabling sulfate-free shampoos to deliver consumer preferred performance from their shampoo.

Liquid crystals are substances that possess mechanical properties resembling those of fluids yet exhibit birefringence under static conditions. If we consider a crystalline solid to have order in all directions, X, Y and Z, then liquid crystals are phases that are ordered or crystalline in only one or two of their three possible orthogonal directions and are disordered (random or liquid-like) in the other dimensions. The presence of lyotropic liquid crystals in the (dilute) shampoo composition can be confirmed by means known to one of skill in the art, such as X-ray analysis and optical microscopy.

The lyotropic liquid crystal forming cationic polymers of the present invention are non-cross-linked polymers. Cross-linked polymers have the back bones of the polymers chemically bound to each other, which forms a 3-dimensional polymer structure. It is believed, without being bound by theory, that lyotropic liquid crystals comprise layers of polymer and surfactant, and thus the polymer needs a certain degree of flexibility to form the liquid crystal phase. The inflexibility of a cross-linked polymer therefore is not preferred. Reference: Chapter 8 “The Aqueous Phase Behavior of Surfactants” by R. G. Laughlin.

The shampoo composition can have a pH of 4 to 8 (e.g., 4.5 to 7.5, 5 to 7, 5.5 to 6.5, 5 to 6, 5.5 to 6, or even 6 to 7), according to the pH Test Method described in the Methods below. Depending on the surfactants and/or the presence of the anti-dandruff active piroctone olamine (PO), there are certain pH ranges that are preferred. For isethionate or sarcosinate containing compositions, pH 5.5-6 or 6-7 may be preferred to minimize surfactant hydrolysis. For shampoo composition containing PO, pH 5-6 is preferred because it helps to boost PO deposition.

The shampoo composition can have a viscosity of 3 Pa-s to 20 Pa-s (e.g., 4 Pa-s to 15 Pa-s, 4.5 Pa-s to 12 Pa-s, 5 Pa-s to 11 Pa-s, and 7 Pa-s to 10 Pa-s) at 26.7° C. according to the Cone/Plate Viscosity Measurement Test Method described hereinbelow.

It may be consumer desirable to have a shampoo composition with a minimal level of ingredients. The shampoo composition can be formulated without polymeric thickeners or suspending agents such as carbomer, EGDS or thixcin. The shampoo composition may be comprised of 9 or fewer ingredients, 8 or fewer ingredients, 7 or fewer ingredients. The minimal ingredient formula can include water, anionic surfactant, amphoteric surfactant, cationic polymer, inorganic salt, and perfume. It is understood that perfumes can be formed from one or more fragrances. In some examples, the composition can be free of or substantially free of fragrance. In another example, the composition can be free of or substantially free of PEG.

The shampoo composition can be used to clean and condition hair. First, the user dispenses the liquid shampoo composition from the bottle into their hand or onto a cleaning implement. Then, they massage the shampoo into their wet hair. While they are massaging the shampoo composition into the hair the shampoo is diluted and a coacervate can form and the shampoo can lather. After massaging into hair, the shampoo composition is rinsed from the user's hair and at least a portion of the cationic polymers can be deposited on the user's hair, which can provide a conditioning benefit. Shampooing can be repeated, if desired, and/or a conditioner can be applied. The conditioner can be a rinse-off conditioner or a leave-in conditioner.

As used herein, “cleansing composition” includes personal cleansing products such as shampoos, conditioners, conditioning shampoos, shower gels, liquid hand cleansers, facial cleansers, and other surfactant-based liquid compositions.

“Clear” or “transparent” can be used interchangeably and mean that the composition has a percent transmittance (% T) of at least 80% at 600 nm (e.g., 80% to 100%).

As used herein, the term “fluid” includes liquids and gels.

As used herein, “molecular weight” or “M.Wt.” refers to the weight average molecular weight unless otherwise stated. Molecular weight is measured using industry standard method, gel permeation chromatography (“GPC”). The molecular weight has units of grams/mol.

“Substantially free” means that a material is present in the composition at less than 0.5 wt % (e.g., less than 0.25%, 0.1%, 0.05%, 0.02%, or even less than 0.01%). “Free of” means that there is no detectable amount of a material present in the composition (i.e., 0 wt %).

“Sulfate-free” and variations thereof means the composition is substantially free of or free of sulfate-containing compounds.

“Sulfated surfactants” or “sulfate-based surfactants” means surfactants that contain a sulfate group.

“Dilution” means at least a 1:1 ratio of composition to water. A composition is diluted when there is a ratio of composition to water 1:1 to 1:20, in some cases 1:1 to 1:10.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials. Unless otherwise noted, all component or composition levels are in reference to the active portion of that component or composition, and are exclusive of impurities, for example, residual solvents or by-products, which may be present in commercially available sources of such components or compositions. It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Surfactant

The cleansing compositions described herein can include one or more sulfate-free surfactants. Surfactants provide a cleaning benefit to soiled articles such as hair, skin, and hair follicles by facilitating the removal of oil and other soils. Surfactants generally facilitate such cleaning due to their amphiphilic nature which allows for the surfactants to break up, and form micelles around, oil and other soils which can then be rinsed out, thereby removing them from the soiled article. The concentration of the sulfate-free surfactant(s) in the composition should be sufficient to provide the desired cleaning and lather performance. For example, the cleansing composition may have a total surfactant level of 5% to 50% (e.g., 8% to 40%, 10% to 30%, 12% to 25%, 13% to 23%, 14% to 21%, 15% to 20%).

The cleansing composition herein includes a surfactant with anionic moieties that can form a coacervate with a suitable cationic polymer. Thus, the surfactants herein can be anionic, amphoteric, zwitterionic, non-ionic, and combinations thereof. Some non-limiting examples of these surfactants are described in U.S. Publication Nos. 2019/0105246 and 2018/0098923, U.S. Pat. No. 9,271,908, and McCutcheon's Emulsifiers and Detergents, 2019, MC Publishing Co.

Suitable anionic surfactants that are substantially free of sulfates can include sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof. The anionic surfactant may be present in the cleansing composition at 3% to 30% (e.g., 4% to 20%, 5% to 15%, 6% to 12%, or even 7% to 10%).

The surfactant system can include one or more amino acid based anionic surfactants. Non-limiting examples of amino acid based anionic surfactants can include sodium, ammonium or potassium salts of acyl glycinates; sodium, ammonium or potassium salts of acyl sarcosinates; sodium, ammonium or potassium salts of acyl glutamates; sodium, ammonium or potassium salts of acyl alaninates and combinations thereof.

Suitable surfactants that are substantially free of sulfates can include sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof. The anionic surfactant may be present in the cleansing composition at 3% to 30% (e.g., 4% to 20%, 5% to 15%, 6% to 12%, or even 7% to 10%).

In some examples, the surfactant system may include one or more amino acid based anionic surfactants. Non-limiting examples of amino acid based anionic surfactants can include sodium, ammonium or potassium salts of acyl glycinates; sodium, ammonium or potassium salts of acyl sarcosinates; sodium, ammonium or potassium salts of acyl glutamates; sodium, ammonium or potassium salts of acyl alaninates and combinations thereof. In some examples, the composition may contain an anionic surfactant selected from the group consisting of sulfosuccinates, isethionates, sulfonates, sulfoacetates, glucose carboxylates, alkyl ether carboxylates, acyl taurates, and combinations thereof.

Some non-limiting examples of sulfosuccinate surfactants are disodium N-octadecyl sulfosuccinate, disodium lauryl sulfosuccinate, diammonium lauryl sulfosuccinate, sodium lauryl sulfosuccinate, disodium laureth sulfosuccinate, tetrasodium N-(1,2-dicarboxyethyl)-N-octadecyl sulfosuccinnate, diamyl ester of sodium sulfosuccinic acid, dihexyl ester of sodium sulfosuccinic acid, dioctyl esters of sodium sulfosuccinic acid, and combinations thereof. Some non-limiting examples of isethionates are sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate, sodium stearoyl methyl isethionate, and mixtures thereof. Some non-limiting examples of sulfonates can include alpha olefin sulfonates, linear alkylbenzene sulfonates, sodium laurylglucosides hydroxypropylsulfonate and combination thereof. Some non-limiting examples of sulfoacetates can include sodium lauryl sulfoacetate, ammonium lauryl sulfoacetate and combination thereof. Some non-limiting examples of glucose carboxylates can include sodium lauryl glucoside carboxylate, sodium cocoyl glucoside carboxylate and combinations thereof. Non-limiting example of alkyl ether carboxylate can include sodium laureth-4 carboxylate, laureth-5 carboxylate, laureth-13 carboxylate, sodium C12-13 pareth-8 carboxylate, sodium C12-15 pareth-8 carboxylate and combination thereof. Non-limiting example of acyl taurates can include sodium methyl cocoyl taurate, sodium methyl lauroyl taurate, sodium methyl oleoyl taurate and combination thereof.

The cleansing composition may include 3% to 40% of an amphoteric surfactant (e.g., 4% to 30%, 5% to 25%, 6% to 18%, 7% to 15%, 8% to 13%, or even 9% to 11%). The ratio of anionic surfactant to amphoteric surfactant can be 0.25:1 to 3:1, 0.3:1 to 2.5:1, 0.4:1 to 2:1, 0.5:1 to 1.5:1, 0.6:1 to 1.25:1, and 0.75:1 to 1:1. In some examples, the ratio of anionic surfactant to amphoteric surfactant is less than 2:1, 1.75:1, 1.5:1, 1.1:1, or even less than 1:1. The e amphoteric surfactant can be selected from betaines, sultaines, hydroxysultanes, amphohydroxypropyl sulfonates, alkyl amphoactates, alkyl amphodiacetates, alkyl amphopropionates and combination thereof.

Some non-limiting examples of betaine amphoteric surfactants include coco dimethyl carboxymethyl betaine, cocoamidopropyl betaine (CAPB), cocobetaine, lauramidopropyl betaine (LAPB), coco-betaine, cetyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, and mixtures thereof. Examples of sulfobetaines can include coco dimethyl sulfopropyl betaine, stearyl dimethyl sulfopropyl betaine, lauryl dimethyl sulfoethyl betaine, lauryl bis-(2-hydroxyethyl) sulfopropyl betaine and mixtures thereof. Some non-limiting examples of alkylamphoacetates amphoteric surfactants include sodium cocoyl amphoacetate, sodium lauroyl amphoacetate and combination thereof. Some particularly suitable examples of amphoteric surfactants are cocamidopropyl betaine (CAPB), lauramidopropyl betaine (LAPB), coco-betaine, cetyl betaine and combinations thereof.

The cleansing composition may include one or more non-ionic surfactants selected from alkyl polyglucosides, alkyl glycosides, acyl glucamides and mixture thereof. Some non-limiting examples of alkyl glucosides include decyl glucoside, cocoyl glucoside, lauroyl glucoside and combination thereof. Some non-limiting examples of acyl glucamide include lauroyl/myristoyl methyl glucamide, capryloyl/caproyl methyl glucamide, lauroyl/myristoyl methyl glucamide, cocoyl methyl glucamide and combinations thereof.

The cleansing composition may include a non-ionic detersive surfactant such as, for example, cocamide, cocamide methyl MEA, cocamide DEA, cocamide MEA, cocamide MIPA, lauramide DEA, lauramide MEA, lauramide MIPA, myristamide DEA, myristamide MEA, PEG-20 cocamide MEA, PEG-2 cocamide, PEG-3 cocamide, PEG-4 cocamide, PEG-5 cocamide, PEG-6 cocamide, PEG-7 cocamide, PEG-3 lauramide, PEG-5 lauramide, PEG-3 oleamide, PPG-2 cocamide, PPG-2 hydroxyethyl cocamide, and mixtures thereof.

Cationic Polymer

The cleansing composition herein includes a cationic polymer that can form a coacervate with the anionic moieties of the surfactant(s). Some non-limiting examples of cationic polymers that may be suitable for use herein include cationic guar polymers, cationic non-guar galactomannan polymers, cationic starch polymers, cationic copolymers of acrylamide monomers and cationic monomers, synthetic non-crosslinked cationic polymers, which may or may not form lyotropic liquid crystals upon combination with the detersive surfactant, and cationic cellulose polymers. Some non-limiting examples of these cationic polymers are disclosed in U.S. Publication Nos. 2019/0105247 and 2021/0346265.

Some particularly suitable examples of cationic guar polymers include guar hydroxypropyltrimonium chloride such as the Jaguar® series from Solvay® S.A., Hi-Care™ series from Rhodia®, and N-Hance™ and AquaCat™ from Ashland™. Some particularly suitable examples of galactomannan polymer derivative include galactomannan polymers that have a mannose to galactose ratio of greater than 2:1 on a monomer to monomer basis obtained from the endosperm of seeds of the Leguminosae family (e.g., tara gum (3 parts mannose/1 part galactose), locust bean or carob (4 parts mannose/1 part galactose), and cassia gum (5 parts mannose/1 part galactose)). Some particularly suitable examples of cationic starch particles include those with a degree of substitution of 0.2 to 2.5 using substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. The “degree of substitution” of the cationically modified starch polymers is an average measure of the number of hydroxyl groups on each anhydroglucose unit which is derivatized by substituent groups. Some particularly suitable examples of cationic cellulose polymers include salts of hydroxyethyl cellulose reacted with a suitable ammonium substituted epoxide such as polyquaternium 10, polyquaternium 24, and polyquaternium 67. Some non-limiting examples of cationic copolymers of acrylamide monomers and cationic monomers include polyquaternium 76 and trimethylammoniopropylmethacrylamide chloride-N-acrylamide (AM:MAPTAC). Another particularly suitable cationic polymer includes polydiallyldimethylammonium chloride, which is sometimes referred to as poly-DADMAC or polyquaternium 6.

The cationic polymer described herein can also aid in repairing damaged hair, particularly chemically treated hair by providing a surrogate hydrophobic F-layer. The microscopically thin F-layer provides natural weatherproofing, while helping to seal in moisture and prevent further damage. Chemical treatments damage the hair cuticle and strip away its protective F-layer. As the F-layer is stripped away, the hair becomes increasingly hydrophilic. It has been found that when lyotropic liquid crystals are applied to chemically treated hair, the hair becomes more hydrophobic and more virgin-like, in both look and feel. Without being limited to any theory, it is believed that the lyotropic liquid crystal complex creates a hydrophobic layer or film, which coats the hair fibers and protects the hair, much like the natural F-layer protects the hair.

The cationic polymer may be present in the cleansing composition at 0.05% to 3% (e.g., 0.075% to 2.0%, 0.1% to 1.0%, 0.16% to 0.5%, 0.2% to 0.5%, 0.3% to 0.5%, or even 0.4% to 0.5%). The cationic polymers may have a cationic charge density of 0.6 meq/g or more (e.g., 0.9 meq/g, 1.2 meq/g, or 1.5 meq/g or more), but typically less than 7 meq/g (e.g., 2 meq/g-7 meq/g, 3 meq/g-6 meq/g, or even 4 meq/g-5 meq/g). In some examples the composition can include a cationic polymer with charge density of 1.7 to 2.1 meq/g and 1 to 1.5% total inorganic salt. The charge densities can be measured at the pH of intended use of the cleansing composition. (e.g., at pH 3 to pH 9; or pH 4 to pH 8). The average molecular weight of cationic polymers can be between 10,000 Da and 10 million Da (e.g., 50,000 Da to 5 million Da, 100,000 Da to 3 million Da, 300,000 Da to 3 million Da, or even 100,000 Da and 2.5 million Da). Lower molecular weight cationic polymers tend to have greater translucency in the liquid carrier of a cleansing composition.

In some instances, the composition may include a cationic polymer system of 2 or more cationic polymers. For example, the cleansing composition may include a primary cationic polymer that has a charge density of 2 meq/gm to 7 meq/gm (e.g., 3 meq/gm to 7 meq/gm, 4 meq/gm to 7 meq/gm, or even 4.5 meq/gm to 7 meq/gm) and one or more secondary cationic polymers that each have a charge density of 0.6 meq/gm to 4 meq/gm (e.g., 0.6 meq/gm to 2 meq/gm). In some instances, the secondary polymers may form an isotropic floc coacervate upon dilution. The charge density of cationic polymers other than cationic guar polymers can be determined by measuring % Nitrogen according to USP <461> Method II. The % Nitrogen can then be converted to Cationic Polymer Charge Density using calculations known in the art. For cationic guar polymers, the charge density is calculated by first calculating the degree of substitution, as disclosed in WO 2019/096601, and then calculate cationic charge density from the degree of substitution, as described in WO 2013/011122.

Liquid Carrier

As can be appreciated, cleansing compositions can desirably be in the form of pourable liquid under ambient conditions. Inclusion of an appropriate quantity of a liquid carrier can facilitate the formation of a cleansing composition having an appropriate viscosity and rheology. A cleansing composition can include, by weight of the composition, 20% to 95%, by weight, of a liquid carrier, and 60% to 85%, by weight, of a liquid carrier. The liquid carrier can be an aqueous carrier such as water.

Optional Ingredients

As can be appreciated, cleansing compositions described herein can include a variety of optional ingredients to tailor the properties and characteristics of the composition. As can be appreciated, suitable optional ingredients are well known and can generally include any ingredients which are physically and chemically compatible with the essential ingredients of the cleansing compositions described herein. Optional ingredients should not otherwise unduly impair product stability, aesthetics, or performance Individual concentrations of optional ingredients can generally range 0.001% to 10%, by weight of a cleansing composition. Optional ingredients can be further limited to ingredients which will not impair the clarity of a translucent cleansing composition.

Suitable optional ingredients which can be included in a cleansing composition can include co-surfactants, deposition aids, conditioning agents (including hydrocarbon oils, fatty esters, silicones), anti-dandruff agents, anti-fungal agents, suspending agents, viscosity modifiers, dyes, nonvolatile solvents or diluents (water soluble and insoluble), pearlescent aids, foam boosters, pediculocides, pH adjusting agents, perfumes, preservatives, chelants, proteins, amino acids, skin active agents, sunscreens, UV absorbers, vitamins, and combinations thereof. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of non-limiting materials that can be added to the composition herein.

Conditioning Agents

The cleansing composition nay include a synthetic conditioning agent (e.g., silicone conditioning agent), an organic conditioning material such as oil or wax, or a combination of these. The silicone conditioning agent can be a volatile silicone, non-volatile silicone, or a combination thereof. Examples of suitable silicone conditioning agents, and optional suspending agents for the silicone, are described in U.S. Reissue Pat. No. 34,584, U.S. Pat. Nos. 5,104,646, 5,106,609, and 11,116,703.

The organic conditioning agent may be non-polymeric, oligomeric or polymeric. Some non-limiting examples of organic conditioning agents include hydrocarbon oils, polyolefins, fatty esters, fluorinated conditioning compounds, fatty alcohols, alkyl glucosides and alkyl glucoside derivatives, quaternary ammonium compounds, polyethylene glycols and polypropylene glycols having a molecular weight of up to 2,000,000 including those with CTFA names PEG-200, PEG-400, PEG-600, PEG-1000, PEG-2M, PEG-7M, PEG-14M, PEG-45M and mixtures thereof.

Emulsifiers

A variety of anionic and nonionic emulsifiers can be used in the cleansing composition of the present invention. The anionic and nonionic emulsifiers can be either monomeric or polymeric in nature. Monomeric examples include, by way of illustrating and not limitation, alkyl ethoxylates, alkyl sulfates, soaps, and fatty esters and their derivatives. Polymeric examples include, by way of illustrating and not limitation, polyacrylates, polyethylene glycols, and block copolymers and their derivatives. Naturally occurring emulsifiers such as lanolins, lecithin and lignin and their derivatives are also non-limiting examples of useful emulsifiers.

Chelating Agents

The cleansing composition can may include 0.01% to 10% of a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996). When related to chelants, the term “salts and derivatives thereof” means the salts and derivatives comprising the same functional structure (e.g., same chemical backbone) as the chelant they are referring to and that have similar or better chelating properties. Some non-limiting examples of chelants that may be suitable for use herein are disclosed in U.S. Pat. Nos. 5,747,440 and 5,284,972. Particularly suitable examples of chelants include polymeric ethylenediaminedisuccinic acid (EDDS) and histidine.

Gel Network

The cleansing composition herein may include a fatty alcohol gel network. Gel networks are formed by combining fatty alcohols and surfactants at a suitable ratio (e.g., 1:1 to 40:1, 2:1 to 20:1, or 3:1 to 10:1). The formation of a gel network involves heating a dispersion of the fatty alcohol in water with the surfactant to a temperature above the melting point of the fatty alcohol. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature, the liquid crystal phase is converted into a solid crystalline gel network. Gel networks can provide a number of benefits to cleansing compositions. For example, a gel network can provide a stabilizing benefit to cosmetic creams and hair conditioners. In addition, gel networks can provide conditioned feel benefits to hair conditioners and shampoos.

Some non-limiting examples of gel networks are disclosed in U.S. Pat. No. 10,912,719. In some examples, a gel network can be prepared by charging a vessel with water. In these examples, the water can then be heated to 74° C. A fatty alcohol (e.g., cetyl alcohol and stearyl alcohol) and a surfactant can be added to the heated water. After mixing, the resulting mixture can passed through a heat exchanger where the mixture is cooled to 35° C., which allows the fatty alcohols and surfactant to crystallize and form a crystalline gel network. Table 1 provides the components and their respective amounts for this example.

TABLE 1

| Premix | % |
|---|---|
| Gel Network Surfactant¹ | 11.00 |
| Stearyl Alcohol | 8% |
| Cetyl Alcohol | 4% |
| Water | QS |

¹For anionic gel networks, suitable gel network surfactants above include surfactants with a net negative charge including sulfonates, carboxylates and phosphates among others and mixtures thereof. For cationic gel networks, suitable gel network surfactants above include surfactants with a net positive charge including quaternary ammonium surfactants and mixtures thereof. For Amphoteric or Zwitterionic gel networks, suitable gel network surfactants above include surfactants with both a positive and negative charge at product usage pH including betaines, amine oxides, sultaines, amino acids among others and mixtures thereof.

### Method of Making a Cleansing Composition

A cleansing composition described herein can be formed similarly to known cleansing compositions. For example, the process of making a cleansing composition can include the step of mixing the surfactant, cationic polymer, and liquid carrier together to form a cleansing composition. Additional information on sulfate-free surfactants and other ingredients that are suitable for shampoo compositions is found at U.S. Pub. Nos. 2019/0105247 and 2019/0105246, incorporated by reference.

### Methods

### 1. Clarity Assessment—Measurement of % Transmittance (% T)

Lack of in situ coacervate can be determined by composition clarity. A composition that does not contain in situ coacervate will be clear, if it does not contain any ingredients that would otherwise give it a hazy appearance.

Composition clarity can be measured by % Transmittance. For this assessment to determine if the composition lacks coacervate, the composition should be made without ingredients that would give the composition a hazy appearance such as silicones, opacifiers, non-silicone oils, micas, and gums or anionic rheology modifiers. It is believed that adding these ingredients would not cause in situ coacervate to form prior to use, however these ingredients will obscure measurement of clarity by % Transmittance.

Clarity can be measured by % Transmittance (% T) using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to ensure no air bubbles are within the sample before the % T is measured by the spectrophotometer at 600 nm. Alternatively, multiple samples can be measured simultaneously by using a spectrophotometer such as the SpectraMax M-5 available from Molecular Devices. Multiple samples are transferred into a 96 well visible flat bottom plate (Greiner part #655-001), ensuring that no air bubbles are within the samples. The flat bottom plate is placed within the SpectraMax M-5 and % T measured using the Software Pro v.5™ software available from Molecular Devices. The composition of the present invention may have a percent transmittance (% T) of at least 80% transmittance at 600 nm.

2. Measures of Lyotropic Liquid Crystals and Improved Hair Performance with their Formation Upon Dilution

The composition does not contain in situ coacervate prior to dilution but rather forms a lyotropic liquid crystal coacervate upon dilution. This allows the product to deliver specific hair benefits while alleviating the need to suspend the coacervate in product with an additional ingredient, that otherwise adds cost and complexity to the formula. Secondarily, the absence of in situ coacervate in neat product offers an opportunity for the formulation to be clear in bottle, thus appealing to a specific consumer segment that only uses clear shampoos. The presence of lyotropic liquid crystals in the (dilute) shampoo composition can be confirmed by means known to one of skill in the art, such as X-ray analysis and optical microscopy.

### X-Ray Diffraction (XRD)

X-ray diffraction can be used to confirm the presence of the characteristic lyotropic liquid crystalline phase present within diluted shampoo of the present invention. XRD uses two techniques, SAXS and WAXS, to identify and differentiate liquid and solid crystalline structures. SAXS (Small Angle X-ray Scattering) is used to confirm the presence of a layered liquid crystal phase, and WAXS (Wide Angle X-ray Scattering) is used to differentiate between Lα (liquid) and Lβ (solid) crystalline structures.

Procedure: SAXS (small angle) data is collected with a Bruker NanoSTAR small-angle x-ray scattering instrument. The micro-focus Cu x-ray tube is operated at 50 kV, 0.60 mA with 550 um ScanTex Pinholes. The sample to detector distance is 107.53 cm and the detector a Vantec2K 2-dimensional area detector. Samples are placed in 2 mm quartz capillaries, sealed and analyzed under vacuum with an analysis time of 600 s

WAXS (wide angle) is collected on a Stoe STADI-MP diffractometer. The generator is operated at 40 kV/40 mA, powering a copper anode long-fine-focus Cu x-ray tube. The diffractometer incorporates an incident-beam curved germanium-crystal monochromator, standard incident-beam slit system, and Mythen PSD detector. Data are collected in transmission mode over a range of 0° to 72° 2θ with a step size of 3° 2θ and 15 seconds per step.

### Light Microscopy

The light microscopy of liquid crystals is described in The Microscopy of Liquid Crystals, Norman Hartshorne, Microscopy Publications, Ltd., Chicago, Ill., U.S.A., 1974. Birefringence occurs in general for mesomorphic states. Methods for microscopic observation and evaluation are discussed in Chapter 1, pp. 1-20, and in Chapter 6, pp. 79-90. A preferred method for determining occurrence of liquid crystals is by observing birefringence (a non-limiting example of which is formation of maltese crosses under cross-polarized light) of thin liquid crystal films between glass slides.

### Light Microscopy

The light microscopy of liquid crystals is described in The Microscopy of Liquid Crystals, Norman Hartshorne, Microscopy Publications, Ltd., Chicago, Ill., U.S.A., 1974. Birefringence occurs in general for mesomorphic states. Methods for microscopic observation and evaluation are discussed in Chapter 1, pp. 1-20, and in Chapter 6, pp. 79-90. A preferred method for determining occurrence of liquid crystals is by observing birefringence (a non-limiting example of which is formation of maltese crosses) of thin liquid crystal films between glass slides or from thin slices of a material under a polarizing microscope.

### 3. Wet Combing Force Method

Hair switches of 4 grams general population or color-treated hair at 8 inches length are used for the measurement. Each hair switch is treated with 4 cycles (1 lather/rinse steps per cycle, 0.1 gm cleansing composition/gm hair on each lather/rinse step, drying between each cycle) with the cleansing composition. Four switches are treated with each shampoo. The hair is not dried after the last treatment cycle. While the hair is wet, the hair is pulled through the fine tooth half of two Beautician 3000 combs. Force to pull the hair switch through the combs is measured by a friction analyzer (such as Instron or MTS tensile measurement) with a load cell and outputted in gram-force (gf). The pull is repeated for a total of five pulls per switch. Average wet combing force is calculated by averaging the force measurement from the five pulls across the four hair switches treated with each cleansing composition. Data can be shown as average wet combing force through one or both of the two combs.

### 4. Deposition Method

Deposition of actives can be measured in vitro on hair tresses or in vivo on panelist's heads. The composition is dosed on a hair tress or panelist head at a controlled amount and washed according to a conventional washing protocol. For a hair tress, the tress can be sampled and tested by an appropriate analytical measure to determine quantity deposited of a given active. To measure deposition on a panelist's scalp, the hair is then parted on an area of the scalp to allow an open-ended glass cylinder to be held on the surface while an aliquot of an extraction solution is added and agitated prior to recovery and analytical determination of a given active. To measure deposition on a panelist's hair, a given amount of hair is sampled and then tested by an appropriate analytical measure to determine quantity deposited of a given active.

### Examples

The following examples further describe and demonstrate embodiments within the scope of the present invention. The examples are given solely for the purpose of illustration and are not to be construed as limitations of the present invention, as many variations thereof are possible without departing from the spirit and scope of the invention.

| | Ex. 1 | Ex. 2 | Ex. 3 |
|---|---|---|---|
| | (wt. %) | (wt. %) | (wt. %) |
| Lauramidopropyl Betaine ¹ | — | — | 2.44 |
| Cocamidopropyl Betaine ² | 9.75 | 9.75 | 7.31 |
| Sodium Cocoyl Isethionate ³ | 6.00 | 6.00 | 6.00 |
| Sodium Lauroyl Sarcosinate ⁴ | 2.50 | 2.50 | 2.50 |
| Polyquaternium-6 ⁵ | 0.05 | 0.55 | 0.04 |
| Water, Preservatives, pH | Q.S. | Q.S. | Q.S. |
| adjusters, Fragrance and | to 100 | to 100 | to 100 |
| Optional Components | | | |
| % Transmittance of | 93 | 96 | 91 |
| Composition | | | |

¹ Mackam DAB-ULS available from Solvay
² Amphosol HCA-HP available from Stepan
³ Hostapon SCI-85 C available from Clariant
⁴ SP Crodasinic LS30/NP MBAL available from Croda
⁵ Flocare C 106 MSS available from SNF

| | Ex. 4 | Ex. 5 | Ex. 6 |
|---|---|---|---|
| | (wt. %) | (wt. %) | (wt. %) |
| Sodium Methyl Cocoyl Taurate ¹ | 12.00 | 10.00 | 9.00 |
| Cocamidopropyl Betaine ² | — | 6.00 | — |
| Coco-betaine ³ | 6.00 | — | 5.00 |
| Polyquaternium-6 ⁴ | 0.4 | 0.1 | 0.25 |
| Piroctone Olamine ⁵ | — | — | 0.5 |
| Water, Preservatives, pH | Q.S. | Q.S. | Q.S. |
| adjusters, Fragrance and | to 100 | to 100 | to 100 |
| Optional Components | | | |
| % Transmittance of | 95 | 97 | 99 |
| composition | | | |

¹ Pureact WS Conc available from Innospec
² Amphosol HCA-HP available from Stepan
³ Dehyton AB 30 available from BASF
⁴ Flocare C 106 MSS available from SNF
⁵ Octopirox available from Clariant

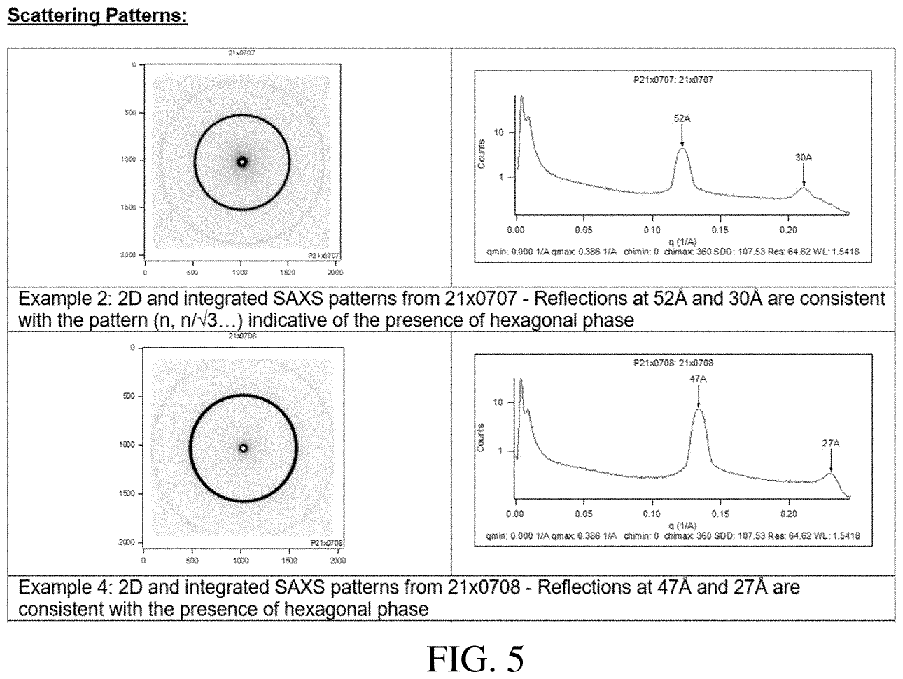

The SAXS patterns collected and summarized in the table below and in FIG. 5 are consistent with the presence of hexagonal liquid crystal phase.

| | X-ray (SAXS) observations of shampoo examples |
|---|---|
| Examples | above @ 10:1 water-to-shampoo dilution |
| 2 | Hexagonal reflections (basal spacing 52 Å) |
| 4 | Hexagonal reflections (basal spacing 47 Å) |

Examples below are presented to further illustrate, but not to limit, the present invention:

| | Ex. 7 | Ex. 8 | Ex. 9 | Ex. 10 |
|---|---|---|---|---|
| | (wt. %) | (wt. %) | (wt. %) | (wt. %) |
| Sodium Methyl Cocoyl Taurate ¹ | 8.00 | 10.00 | 7.00 | — |
| Cocamidopropyl Betaine ² | 4.00 | — | 6.00 | 7.31 |
| Coco-betaine ³ | — | 4.00 | — | — |
| Cetyl betaine ⁴ | — | 2.00 | — | — |
| Lauramidopropyl Betaine ⁵ | — | — | — | 2.44 |
| Sodium Cocoyl Isethionate ⁶ | — | — | — | 6.00 |
| Sodium Lauroyl Sarcosinate ⁷ | — | — | — | 2.50 |
| Polyquaternium-6 ⁸ | 0.05 | 0.1 | 0.2 | 0.25 |
| Water, Preservatives, pH | Q.S. | Q.S. | Q.S. | Q.S. |
| adjusters, Fragrance and | to 100 | to 100 | to 100 | to 100 |
| Optional Components | | | | |

¹ Pureact WS Conc available from Innospec
² Amphosol HCA-HP available from Stepan
³ Dehyton AB 30 available from BASF
⁴ Amphosol CDB-HP available from Stepan
⁵ Mackam DAB-ULS available from Solvay
⁶ Hostapon SCI-85 C available from Clariant
⁷ SP Crodasinic LS30/NP MBAL available from Croda
⁸ Flocare C 106 MSS available from SNF

| | Ex. 11 | Ex. 12 | Ex. 13 | Ex. 14 |
|---|---|---|---|---|
| | (wt. %) | (wt. %) | (wt. %) | (wt. %) |
| Sodium Methyl Cocoyl Taurate ¹ | 12.00 | 10.00 | — | — |
| Cocamidopropyl Betaine ² | — | 5.00 | 9.75 | 7.31 |
| Coco-betaine ³ | 6.00 | — | — | — |
| Lauramidopropyl Betaine ⁴ | — | — | — | 2.44 |
| Sodium Cocoyl Isethionate ⁵ | — | — | 6.00 | 6.00 |
| Sodium Lauroyl Sarcosinate ⁶ | — | — | 2.50 | 2.50 |
| Polyquaternium-10 ⁷ | — | — | 0.5 | — |
| Polyquaternium-10 ⁸ | — | — | — | 0.5 |
| Polyquaternium-10 ⁹ | 0.5 | — | — | — |
| Polyquaternium-10 ¹⁰ | — | 0.5 | — | — |
| Water, Preservatives, pH | Q.S. | Q.S. | Q.S. | Q.S. |
| adjusters, Fragrance and | to 100 | to 100 | to 100 | to 100 |
| Optional Components | | | | |

¹ Pureact WS Conc available from Innospec
² Amphosol HCA-HP available from Stepan
³ Dehyton AB 30 available from BASF
⁴ Mackam DAB-ULS available from Solvay
⁵ Hostapon SCI-85 C available from Clariant
⁶ SP Crodasinic LS30/NP MBAL available from Croda
⁷ Cationic Cellulose 21251-46: MW = 2,000,000 g/mol, CD = 2.9 meq/g available from Dow
⁸ Cationic Cellulose 2298-24D: MW = 2,000,000 g/mol, CD = 2.3 meq/g available from Dow
⁹ Cationic Cellulose 21143-13: MW = 450,000 g/mol, CD = 2.7 meq/g available from Dow
¹⁰ Cationic Cellulose 2298-24B: MW = 2,000,000 g/mol, CD = 2.6 meq/g available from Dow

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A shampoo composition comprising:
a) 3% to 35% of an anionic surfactant; wherein the anionic surfactant is substantially free of sulfated surfactants;
b) 3% to 15% of an amphoteric surfactant; and
c) 0.01% to 2% of a cationic polymer having a charge density 2.0 to 10.0 meq/g, wherein the composition is isotropic and forms a lyotropic liquid crystal coacervate upon dilution.
d) The composition of claim 1, wherein the cationic polymer has a charge density 4.5 to 7.0 meq/g.

2. The composition of claim 1, wherein the shampoo composition is clear and comprises a composition having a % T value of greater than 80.

3. The composition of claim 1, wherein the anionic surfactant is selected from sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

4. The composition of claim 1, wherein the cationic polymer is selected from cationic guars, cationic cellulose, cationic synthetic homopolymers, cationic synthetic copolymers, and combinations thereof.

5. The composition of claim 1, wherein the cationic polymer is selected from cationic synthetic homopolymers, cationic synthetic copolymers, and combinations thereof.

6. The composition of claim 1, wherein the amphoteric surfactant is selected from betaines, sultaines, hydroxysultanes, amphohydroxypropyl sulfonates, alkyl amphoactates, alkyl amphodiacetates, and combination thereof.

7. The composition of claim 1, further comprising an antidandruff agent.

8. The composition of claim 8, wherein the antidandruff agent is selected from piroctone olamine, zinc pyrithione, sulfur, selenium sulfide and azoxystrobin, and combinations thereof.

9. The composition of claim 1, wherein the composition is substantially free of silicones.

10. The composition of claim 1, wherein the composition consists of 9 or fewer ingredients.

11. The composition of claim 1, wherein the cationic polymer is DADMAC.

12. The composition of claim 1, further comprising one or more secondary cationic polymers selected from cationic guars, cationic cellulose, cationic synthetic homopolymers, cationic synthetic copolymers, and combinations thereof, which in combination with the anionic surfactant form an isotropic floc coacervate upon dilution.

13. A method for cleaning hair comprising:
a) providing the shampoo composition of claim 1;
b) dispensing the shampoo composition into a palm or cleaning implement;
c) applying the shampoo composition onto wet hair and massaging the shampoo composition across the hair and scalp; wherein the shampoo composition is diluted forming a lyotropic liquid crystal coacervate that is deposited onto the hair; and
d) rinsing the shampoo composition from the hair.
